(19) United States
(12) Patent Application Publication
(10) Pub. No.: US 2009/0325157 A1
(43) Pub. Date: Dec. 31, 2009

(54) PATHOGEN DETECTION AND SCREENING

(76) Inventor: Crystal R. Icenhour, Palmyra, VA (US)

Correspondence Address: K&L Gates LLP, P.O. BOX 1135, CHICAGO, IL, 60690, US

(21) Appl. No.: 12/119,250

(22) Filed: May 12, 2008

(51) Int. Cl. C12Q 1/68; C07H 21/00

(52) U.S. Cl. 435/6; 536/23.1

(57) ABSTRACT

Disclosed is a rapid dual purpose PCR based method for identifying two or more pathogens in a sample, such as a stool or environmental (soil, water) sample, including Giardia and/or Cryptosporidium in a single real-time PCR reaction. This is of particular utility in the screening and detection of pathogen(s) in water, soil, and/or other environmental applications, as well as in stool sample testing/screening. The present methods are more sensitive than conventional ELISA or IFA microscopic bead methods of detection. The present methods have utility in the detection/screening of these and other pathogens in human an non-human (veterinary and environmental) applications. An internal control construct (ICC) for use in the PCR based nucleic acid detector method is also disclosed.

### (1) GOVERNMENT INTEREST

The United States Government may own rights in the present invention pursuant to NIH 1 R41AI069598-01 and/or 2 R42AI069598-02.

### BACKGROUND

(1) Field of the Invention

The present invention relates to a multiplex PCR/PCR method, which enables in a single assay the simultaneous detection of any combination of pathogens, particularly Giardia and Cryptosporium.

(2) Description of the Related Art

Giardia is a protozoan parasite that is a major cause of diarrhea worldwide. The most common species of Giardia is G. lamblia, which is the most common pathogenic parasite in North America (Meyer and Jarrol (1980) Am. J. Epidemiol. 3: 1-12). Giardia has two life stages. The trophozoite stage inhabits the small intestine of host animals, moving about using flagella. A suction disk allows the trophozoite to attach to the wall of the intestine while it feeds on mucous secretions. The second life stage, the cyst, has a stronger outer layer, and thus better able than the trophozoite to survive outside of the host while passing from host to host. Transmission is typically through Giardia-contaminated water supplies (Meyer and Jarrol, supra.), or person to person (Black et al. (1977) Pediatrics 60: 486-491).

The cytoskeleton of G. lamblia trophozoites contain a group of 29-38 kDa proteins known as giardins (Peattie et al. (1989) J. Cell Biol. 109: 2323-2335). Nucleic acid sequences are known for several of the giardins, including .alpha.-1-giardin and .alpha.-2-giardin, which are 81% identical at the nucleic acid level and have amino acid sequences that are 77% identical (Alonso and Peattie (1992) Mol. Biochem. Parasitol. 50: 95-104). The .alpha.-1-giardin has been identified on the membrane and disk of G. lamblia trophozoites (Wenman et al. (1993) Parasitol. Res. 79: 587-592).

Traditionally, Giardia infection is diagnosed by microscopic detection of ova and parasites (O&P) in stools, which is a laborious process. More recently developed methods for Giardia diagnosis include serologic tests for anti-Giardia antibodies. Little correlation was found, however, between the presence of anti-Giardia antibodies in the serum and active Giardia infection. Other diagnostic methods involve detection of Giardia antigens in stool samples. For example, Green et al. discuss the use of an affinity-purified antiserum raised by inoculating rabbits with whole trophozoites or disrupted trophozoites and cysts (Green et al. (1985) Lancet 2: 691-693). Other groups have described the use of monospecific antibodies that bind to a 65 kDa antigen that is shed in the stool of Giardia Giardiasis patients (Rosoff and Stibbs (1986) J. Clin. Microbiol. 24: 1079-1083; U.S. Pat. No. 5,503,983; Stibbs (1989) J. Clin. Microbiol. 27: 2582-2588; Rosoff et al. (1989) J. Clin. Microbiol. 27: 1997-2002). Monoclonal antibodies that bind to two species of Giardia cyst wall constituents are discussed in Lujan et al. (1995) J. Biol. Chem. 270: 29307-29313. ELISA assays for G. lamblia are discussed in, for example, Nash et al. (1987) J. Clin. Microbiol. 25: 1169-1171; Stibbs et al. (1988) J. Clin. Microbiol. 26: 1665-1669; Ungar et al. (1984) J. Infect. Dis. 149: 90-97.

Previously described assays for detecting Giardia infection often have shortcomings. For example, the assay of Ungar et al. was reported to fail to detect 8% of positive samples, and cannot be read by direct visual inspection (Green et al., supra.).

Giardia lamblia is the only species of the genus that is known to cause disease in humans. Some controversy still surrounds the systematics of the species which is also referred to as Giardia duodenalis or Giardia intestinalis (Lu et al. 1998 Molecular comparison of Giardia lamblia isolates. Int. J. Parasitol. 28: 1341-1345). Other representatives of the genus Giardia described to date are Giardia agilis from amphibians and Giardia muris from rodents, birds and reptiles (Meyer 1994 Giardia as an organism. P 3-13. In: RCA. Thompson, J. A. Reynoldsen, A. J. Lymbery (eds.) Giardia: From molecules to disease. CAB International, Wallingford, Oxon, UK), Giardia ardea from herons (Erlandsen et al. 1990 Axenic culture and characterization of Giardia ardea from the great blue heron (Ardea herodias). J. Prasitol. 76: 717-724) and Giardia microti from muskrats and voles (van Keulen et al. 1998). The sequence of Giardia small subunit rRNA shows that voles and muskrats are parasitized by a unique species Giardia microti. J. Parasitol. 84: 294-300). Monoclonal antibodies (mabs) are the most important and widely applied tool for detection of Giardia cysts in water samples. The vast majority of commercially available antibodies show a lack of specificity as the antibodies detect all Giardia spp including species that do not infect humans. As a positive antibody reaction does not allow any conclusion regarding the viability (infectivity) of the cysts, viability stains (DAPI, PI) have to be used in conjunction with antibodies.

Cryptosporidium parvum is detected by light microscopic examination of fecal smears for oocysts or by PCR of fecal samples using Cryptosporidium parvum specific oligonucleotide primers. For example, U.S. Pat. No. 5,770,368 to De Leon et al. discloses a method for detecting encysted forms of Cryptosporidium that are viable and infectious. The method involves isolating oocysts, inducing transcription of the heat shock protein (HSP) genes, and detecting the induced transcripts by RT-PCR. Alternatively, infectivity is determined by cultivating the Cryptosporidium on susceptible cells and either amplifying HSP DNA from infected cells by PCR or induce HSP transcription and detecting the induced transcripts by RT-PCR.

PCR is generally considered the most sensitive and rapid method for detecting nucleic acids of a pathogen in a particular sample. PCR is well known in the art and has been described in U.S. Pat. No. 4,683,195 to Mullis et al., U.S. Pat. No. 4,683,202 to Mullis, U.S. Pat. No. 5,298,392 to Atlas et al., and U.S. Pat. No. 5,437,990 to Burg et al. In the PCR step, oligonucleotide primer pairs for each of the target pathogens are provided wherein each primer pair comprises a first nucleotide sequence complementary to a sequence flanking the 5′ end of the target nucleic acid sequence and a second nucleotide sequence complementary to a nucleotide sequence flanking the 3′ end of the target nucleic acid sequence. The nucleotide sequences comprising each oligonucleotide primer pair are specific to particular pathogen to be detected and do not cross-react with other pathogens.

There are multiple non-interchangeable real time PCR platforms in use in clinical laboratories. Some, such as the Roche COBAS and HIV RNA amplification machines, are closed platform, sample-in-result-out devices. By design, these are inflexible and not amenable to adapting to other purposes (such as a de novo Giardia or Cryptosporidium assay).

The LightCycler 1.5 (and the updated version, LightCycler 2.0) is the major open platform machine in use in clinical laboratories. It is logical to develop the assay onto a carefully chosen few to increase usability. Therefore, in addition to the LightCycler assay, an assay should also be adaptable to the Cepheid SmartCycler and Applied Biosystems ABI7300/7500. Other candidates are the Corbett Roto-gene and the BioRad iCycler.

None of the above methods are suitable for the simultaneous detection of multiple pathogens in a sample. PCR is a sensitive and rapid method for detecting pathogens, and it is amenable to simultaneously detecting multiple pathogens in a sample. However, using PCR for the simultaneous detection of multiple pathogens in a sample has been problematic. The primary obstacles to simultaneous detection of multiple pathogens have been cross-reactivity and preferential amplification of particular target sequences in the sample at the expense of the other target sequences in the sample.

While U.S. Pat. No. 5,756,701 to Wu et al. discloses a multiplex PCR method for simultaneously detecting Salmonella spp., Yersinia spp., and Escherichia coli in a sample, the method is specific only for the aforementioned bacterial species. U.S. Pat. No. 5,882,856 to Shuber also discloses a multiplex PCR method; however, the method uses chimeric primers comprising a sequence complementary to the target sequence covalently linked to a non-complementary sequence. Franck et al., J. Clin. Microbiol. 36: 1795-1797 (1998), discloses a multiplex PCR method for detecting particular Escherichia coli strains that encode K99 pili or heat-stable enterotoxin STa. In general, because of the difficulty in developing PCR methods that enable simultaneous detection of multiple pathogens in a sample, most samples to be analyzed by PCR for multiple pathogens are separately tested for each of the multiple pathogens in separate PCR reactions.

A PCR assay used by a clinical laboratory needs to have an internal control DNA template that amplifies to confirm that overwhelming PCR inhibition did not occur. This is particularly critical for a stool-based assay due to the complexity of this specimen. The chemistries and fluorophores of an internal control must also be platform-appropriate, and therefore incorporation of an internal control will also take place in this aim.

Currently-used clinical diagnostic and water quality tests for Giardia and Cryptosporidium are time-consuming, difficult to perform, and not as sensitive or specific as desired. Additionally, current tests for detecting Giardia and Cryptosporidium are individual tests. Diagnostic labs use ELISA and/or IFA microscopic identification to diagnose Cryptosporidium and Giardia. Unfortunately, ELISA can only detect one of these two pathogens in a single test. Additionally, the test can take more than 4 hours to perform. IFA microscopy is costly (and involves significant technician time), and provides an unsatisfactory limit of detection (low sensitivity).

In water quality testing, most labs use high volume filtration combined with IFA microscopy. These tests costs are quite high, and also require highly skilled personnel for accurate interpretation of the microscopy. These tests can take up to 2 days to complete.

Because current methods for detecting important infectious agents requires performing separate assays, there is a need for a method which would enable the simultaneous detection of two or more disease-causing or linked pathogens. Simultaneous detection would provide substantial savings in cost and time in identifying specific infectious agents associated with a given human or other animal disease outbreak. Simultaneous detection of two or more infectious pathogens in a single assay would avoid the potential for overlooking dual infections, and permit early and appropriate therapy initiation in a timely and more effective manner.

### SUMMARY

The present invention, in a general and overall sense, provides a unique method for detecting multiple pathogens and/or other contaminants in a sample containing a biological specimen using a single assay. In some aspects, the method provides for the detection of Giardia and/or Cryptosporidium in a single, real-time PCR reaction. Among other features, this provides for a very sensitive and specific method having a multiplex capacity for pathogen detection in a single step method. The methods are therefore important in many applications, including clinical diagnosis of animal (human and non-human) pathologies and environmental (water and soil) screening/testing contaminant identification.

According to some embodiments, a biological specimen may include virtually any specimen capable of containing a pathogenic organism, such as G. lamblia, Cryptosporidium, Salmonela, Shigella, Campylobacter, Candida, E. coli, Yersinia, Aeromonas, or other small parasitic organism. A biological specimen may comprise a sample obtained from a water supply, sewer treatment area, a soil sample from a farming area, animal grazing area, waste disposal area, and/or a sample obtained from virtually any water source used by animals or humans for consumption, cleaning or any other domestic or commercial use. In addition, a biological sample may comprise human or animal waste materials (e.g., stool), medical refuse (bandages and wound dressings), and/or body fluid (urine, plasma, blood, mucus, etc). In some embodiments, the methods provide for the screening and/or testing of a biological specimen such as drinking water and/or bodies of water (such as a stream, river, or lake) from which drinking water is obtained.

In some aspects, there is provided a pathogen detection and screening method that is 50% or more less time consuming than conventional methods for pathogen detection in measuring the same or similar pathogen. In some embodiments, the methods are also significantly less expensive than currently available methods. By way of example, the method is about 35% less expensive than currently available detection methods used for similar purposes, such as ELISA or microscopic examination methods.

In another aspect, there is provided a method that is capable of genetically detecting two or more microorganisms in a sample simultaneously. By way of example, such two or more microorganisms may comprise Giardia and Cryptosporidium. Among other things, this multiplex measurement and detection feature provides an advantage of providing a single test, while conventional methods require two or more individual tests for providing the same clinical and/or screening detection result.

The present methods also provide for a protocol that takes only about 2 hours for detection, is more sensitive, is more specific, and does not require interpretation of results. In some specific embodiments, the methods provide for water quality testing. These types of testing typically require a relatively high volume filtration. Because the present analytical tests and methods rely on real-time PCR detection, which detects microorganism specific (e.g., Cryptosporidium- and/or Giardia-specific) DNA sequences, a relatively high volume filtration may not be needed. In contrast to other forms of water quality testing, the present methods do not rely on visual determination or antibody binding.

Commercial uses of the present methods include clinical diagnosis of an animal (human or non-human) stool specimen, veterinary diagnosis from animal stool specimen, water quality testing from recreational and/or drinking water samples, and environmental testing from soil or other potentially contaminated samples.

The present invention provides a multiplex PCR/PCR assay which enables in a single assay the simultaneous detection of Giardia and Cryptosporidium parvum. The present invention has the advantage over the prior art in that it can detect any combination of two (2) or more infectious agents, such as Giardia and Cryptosporidium, in a single assay without the use of antibodies (i.e., in traditional ELISA methodologies).

In one aspect, a nucleic acid-based screening/detection method capable of simultaneously detecting two or more pathogens (multiplex assay), such as Cryptosporidium and Giardia, in a biological sample, such as a fecal sample, is provided. In one embodiment, the method comprises: (a) isolating a nucleic acid sample (DNA) from a biological (e.g., stool) sample to provide an isolated test nucleic acid sample; (b) combining in a PCR reaction mixture said isolated test nucleic acid sample with at least two primer pairs selected from the group consisting of a first oligonucleotide primer pair that is capable of hybridizing to opposite strands of a target nucleic acid sequence, such as a target nucleic acid sequence of Cryptosporidium, a second oligonucleotide primer pair that is capable of hybridizing to opposite strands of a second target nucleic acid sequence, such as a target nucleic acid sequence of Giardia, and a third oligonucleotide primer pair that is capable of hybridizing to opposite strands of an internal control target nucleic acid sequence, wherein each primer pair flanks its target nucleic acid sequence for PCR amplification of the target nucleic acid sequence, and wherein the PCR mixture comprises four deoxynucleotide triphosphates selected from the group consisting of adenosine deoxynucleotide triphosphate, guanosine deoxynucleotide triphosphate, thymidine deoxynucleotide triphosphate, cytosine deoxynucleotide triphosphate, and nucleotide analogs thereof, and a thermostable DNA polymerase; (c) synthesizing a target DNA comprising the nucleic acids with the deoxynucleotide triphosphates; (d) amplifying the target DNA in the reaction mixture under suitable PCR reaction mixture temperature conditions by a repetitive series of PCR thermal cycling steps comprising: (1) denaturing the target DNA and cDNA into opposite strands; (2) hybridizing the oligonucleotide primers to the appropriate denatured strands, and (3) extending the hybridized primers with the four deoxynucleotide triphosphates and the nucleic acid polymerase; and (c) following amplification of the target nucleic acid sequence by one or more series of the thermal cycling steps, screening for the amplified PCR products.

In another aspect, a nucleic acid based screening method for detecting one or more pathogenic microorganisms is provided. (Singleplex assay).

In some embodiments of the method, the predenaturation at 95° C. for 15 minutes. In some embodiments, the PCR reaction is for 44-50 cycles, wherein each cycle consists of denaturing the DNA at about 94° C. for about 30 seconds, annealing the primers to the denatured DNA at about 55° C. for about 30 seconds, and extending the primers at about 72° C. for about 1 minute. Exact temperatures for denaturation, annealing, and extension are unique for each singleplex and/or multiplex assay.

In another aspect, an internal control construct (ICC) is provided. In some embodiments, the ICC construct is a double stranded structure. In some embodiments, the ICC may be described as having the structure:

### Internal Control Construct (ICC):

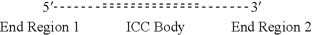

In some embodiments, the ICC structure comprises an ICC body, an end region 1 and an end region 2. The end region 1 and the end region 2 may comprise the same or different base pair sequences. The end region 1 and end region 2 may in some embodiments comprise a sequence that corresponds to the base pair sequence of a primer sequence of a target microorganism to be detected according to the PCR techniques described herein.

In some embodiments, of the above ICC structure, the ICC Body region may be described as having a length of about 190 to about 210 base pairs (bp). In some embodiments, the ICC body may be described as having a length of 207 bp. In one particular embodiment of the ICC, the ICC body will comprise a sequence as defined by the following 207 bp sequence:

SEQ ID NO: 1:
5′-GAA GTT AGT AGT GCG ATC CTT TCT GAC TTT TGT CGT
GCT GTG ACG GTG CTT GCC ATG CGA A C A GCT GCA CAG
GTA CTC GAG GGA AGG CAC GTA AAT TTA GTC CCC CAA
TAA ATA ACA GGC CGC TGT TGA GCA CAA GCA GCT AGC
GCC GTT TTA GCC ACA TGT ACC CAG TAT ATA TGT CAC
GAG AGG ATA GGC GAA TTG GAA TGG TCA GGC C-3′

In some embodiments of the ICC structure, the end region 1 is described as a sequence located at the 5′ end of the structure. The end region one may be further described as a sequence comprising 15 base pairs (bp) to 30 base pairs (bp). In some embodiments, the end region 1 is a sequence of 17 bp. Solely for purposes of example, the end region 1 may comprise a sequence of a primer sequence as described herein as a forward primer for Cryptosporidium. In some embodiments, the specific sequence of the end region 1 having a length of 17 bp is

| | |
|---|---|
| 5′-GCC TAC CGT GGC AAT GA-3′. | (SEQ ID NO: 2) |

By way of further example, the end region 1 may comprise a sequence of a primer sequence as described herein as a forward primer for Giardia. In these embodiments, the specific sequence of the end region 1 that corresponds to a forward primer for Giardia posses a length of 17 bp, and has a sequence of Giardia Forward (primer 1)

| | |
|---|---|
| 5′-GGA CGG CTC AGG ACA AC-3′ | (SEQ ID NO: 3) |

In some embodiments of the ICC structure, the end region 2 is described as a sequence located at the 3′ end of the structure. The end region two (2) may be further described as a sequence comprising 15 base pairs (bp) to 30 base pairs (bp). In some embodiments, the end region 2 is a sequence of 26 bp. Solely for purposes of example, the end region 2 may comprise a sequence of a primer sequence as described herein as a reverse primer for Cryptosporidium In some embodiments, the specific sequence of the end region 2 having a length of 26 bp is:

(SEQ ID NO: 4)

5′-AAA GTC CTG TAT TGT TAT TTC TTG TC-3′

By way of further example, the end region 2 may comprise a sequence of a primer sequence as described herein as a reverse primer for Giardia. In these embodiments, the specific sequence of the end region 2 that corresponds to a reverse primer for Giardia posses a length of 19 bp, and has a sequence of Giardia reverse primer 1:

| | |
|---|---|
| Giardia Reverse (primer 2) | |
| 5′-GGA GTC GAA CCC TGA TTC T-3′. | (SEQ ID NO: 5) |

The term “amplification” of DNA as used herein means the use of polymerase chain reaction (PCR) to increase the concentration of a particular DNA sequence within a mixture of DNA sequences. The particular DNA sequence that is amplified is described herein as a “target” sequence.

In another aspect, a plasmid construct that comprises the Internal Control Construct inserted into the plasmid is provided. This plasmid construct, in one embodiment, is shown at FIG. 6 (pJ201+insert, 2759 bp). In the FIG. 6, the blurred region of the construct corresponds to the ICC bp segment.

The term “primer pair” means a pair of oligonucleotide primers which are complementary to the sequences which flank the target sequence. The primer pair consists of an upstream primer which has a nucleic acid sequence that is complementary to a sequence upstream of the target sequence and a downstream primer which has a nucleic acid sequence that is complementary to a sequence downstream of the target sequence.

The term “multiplex PCR” as used herein means the simultaneous PCR amplification of two (2) or more (e.g., multiple) DNA target sequences in a single mixture.

The term “internal control” sequence as used in the description of the present methods and compositions relates to a nucleic acid sequence that demonstrates the PCR reaction is functioning to detect nucleic acid sequence, and is free of interfering materials in the reaction mixture. The internal control sequence comprises an internal control body segment that comprises a random sequence created by the present investigators and found to be useful in providing an accurate control function.

The primer pairs are provided in particular concentrations that reduce the occurrence of preferential amplification, an undesirable phenomenon characteristic of other methods in PCR reactions which attempt to simultaneously amplify multiple species of target nucleic acid sequences. Preferential amplification results in the disproportionate amplification of one or more target nucleic acid sequence species at the expense of another (e.g., second) target sequence species such that the amount of the preferentially amplified sequences greatly exceeds the amount of the other (e.g., second) non-preferred sequences. The overproduction of amplified product for a particular target sequence species causes the underproduction of amplified product for the other (e.g., second) target sequence species. Thus, a particular target sequence species may not be detectable in a multiplex PCR reaction, even though it is present in the PCR reaction mixture. Preferential amplification occurs, among other reasons, because different primers have different physical properties and, therefore, will have different amplification efficiencies under particular simultaneous PCR reaction conditions.

In addition to the physical characteristics of the primers, other reaction conditions such as magnesium concentration, the type of DNA polymerase used, the concentration of DNA polymerase, the target sequence concentration, annealing temperature, and the primer concentration also affect amplification efficiency of a particular target nucleic acid sequence. In addition, the source from which the target sequences are isolated, e.g., stool (feces) or urine, and the method for isolating the nucleic acids can also affect amplification of particular target sequences.

Because of the large number of variables that need to be adjusted to enable the simultaneous amplification of multiple target nucleic acid sequence species, developing a multiplex PCR method is difficult and time consuming, particularly, when the reaction must further include a preceding reverse transcription step to make the target nucleic acid sequence. In some cases, suitable PCR reaction conditions, which allow the simultaneous amplification of all the target sequence species in the reaction mixture, have remained elusive. Therefore, methods for identifying multiple target nucleic acid sequences typically have required the performance of multiple PCR reactions, wherein each PCR reaction separately detects one of the multiple target nucleic acid sequences in a sample. The present techniques, methods and compositions discover the proper conditions for simultaneously detecting multiple target nucleic acid sequences in a single reaction.

### BRIEF DESCRIPTION OF THE FIGURES

FIG. 1, according to one embodiment of the invention, relates to Cryptosporidium amplification of Cryptosporidium control DNA and Cryptosporidium/Giardia mixed DNA. Channel 2 Amplification Curves detecting LC 640 Red fluorescence by cycle number. All reactions use the standard PCR recipe with both Giardia and Cryptosporidium primers and probes. Blue diamonds are reactions with Giardia DNA added, green squares are with Cryptococcus DNA added, black lines are mixed Giardia and Cryptococcus DNA added, and blue squares are negative (no template) controls. Run in multiplex with Giardia.

FIG. 2, according to one embodiment of the invention, demonstrates Cryptosporidium amplification of Cryptosporidium control DNA and Cryptosporidium/Giardia mixed DNA. Channel 2 Melt Curves detecting LC 640 Red fluorescence with respect to temperature. All reactions use the standard PCR recipe with both Giardia and Cryptosporidium primers and probes. Blue diamonds are reactions with Giardia DNA added, green squares are with Cryptococcus DNA added, black lines are mixed Giardia and Cryptococcus DNA added, and blue squares are negative (no template) controls. Run in multiplex with Giardia.

FIG. 3, according to one aspect of the invention, relates to: Giardia amplification of Giardia control DNA and Cryptosporidium/Giardia mixed DNA. Channel 3 Amplification Curves detecting LC 705 Red fluorescence with respect to temperature. All reactions use the standard PCR recipe with both Giardia and Cryptosporidium primers and probes. Blue diamonds are reactions with Giardia DNA added, green squares are with Cryptococcus DNA added, black lines are mixed Giardia and Cryptococcus DNA added, and blue squares are negative (no template) controls. Run in multiplex with Cryptococcus.

FIG. 4, according to one embodiment of the invention, relates to Giardia amplification of Giardia control DNA and Cryptosporidium/Giardia mixed DNA. Channel 3 Melt curves detecting LC 705 Red fluorescence with respect to cycle number. All reactions use the standard PCR recipe with both Giardia and Cryptosporidium primers and probes. Blue diamonds are reactions with Giardia DNA added, green squares are with Cryptococcus DNA added, black lines are mixed Giardia and Cryptococcus DNA added, and blue squares are negative (no template) controls. Run in multiplex with Cryptococcus.

FIG. 5, according to one embodiment of the invention, provides the general structure of the Internal Control Construct (ICC).

FIG. 6, according to one embodiment of the invention, provides the general structure of a plasmid into which the internal control construct has been inserted. The plasmid here is pJ201+insert, and has a total size of 2,759 bp+insert.

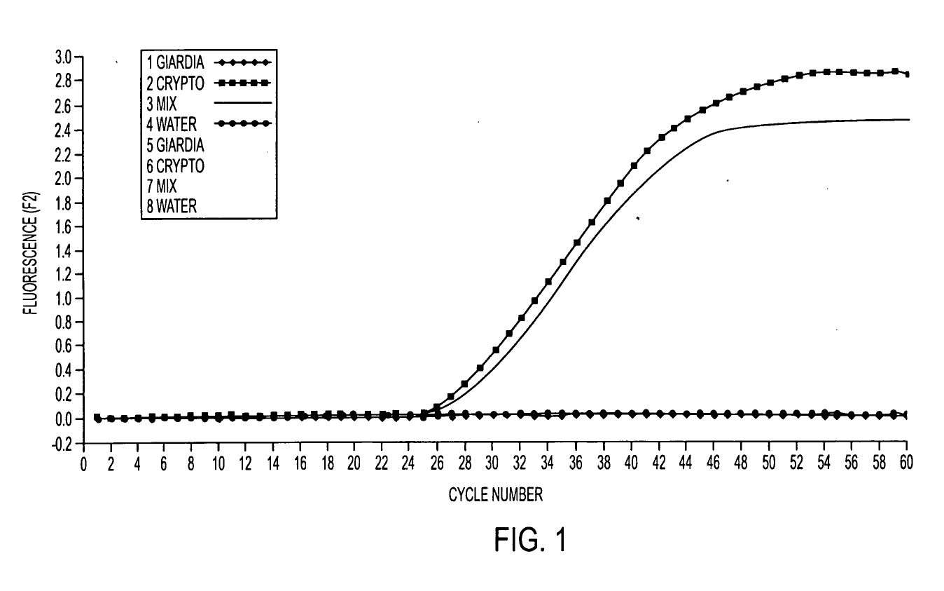

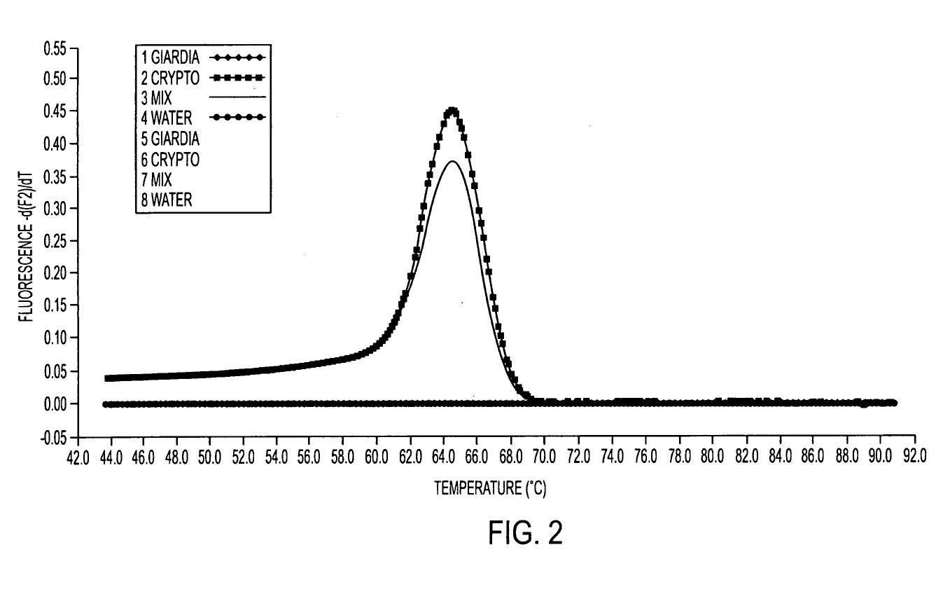

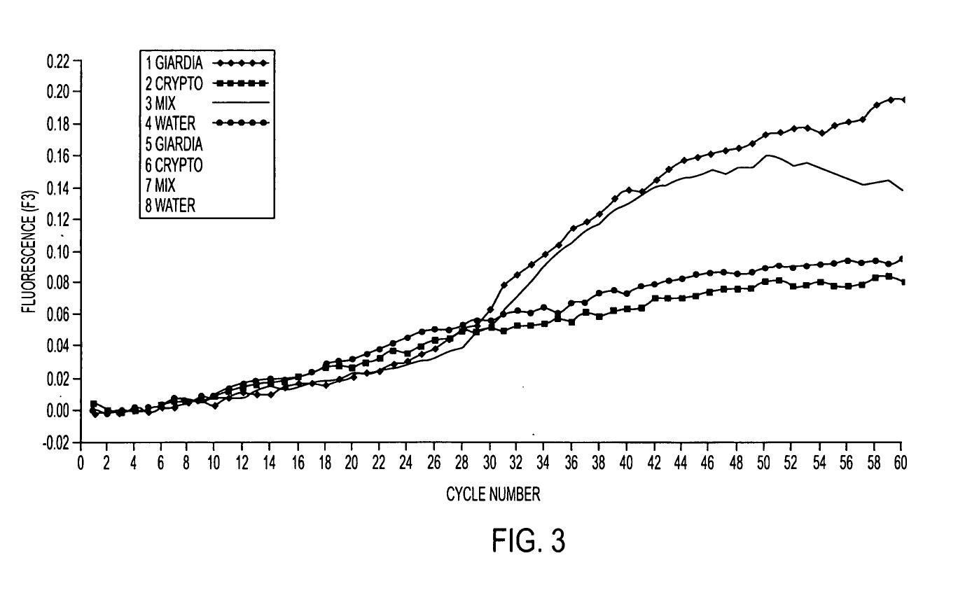

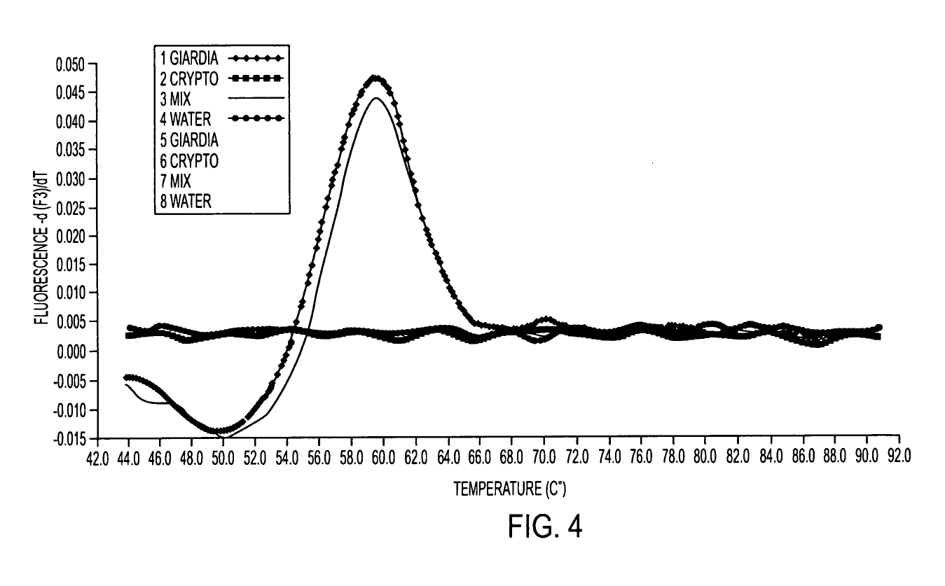

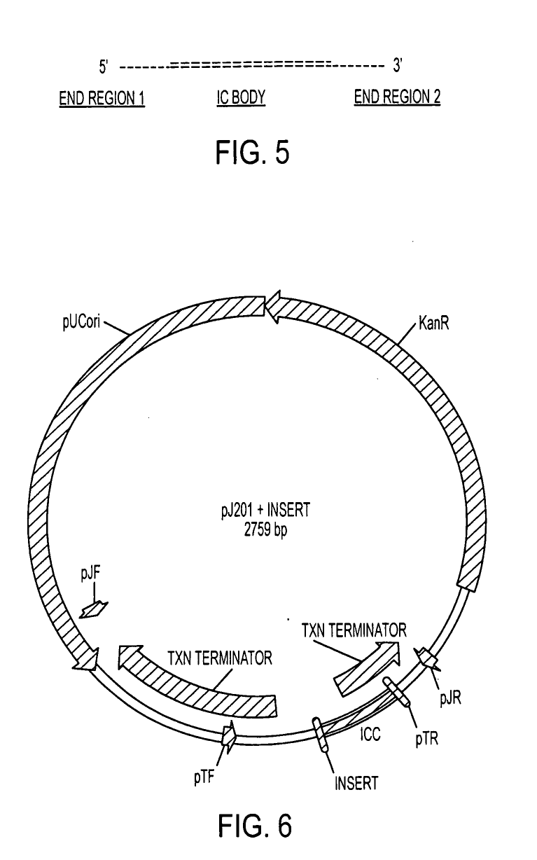

### DETAILED DESCRIPTION OF THE PREFERRED EMBODIMENTS

The present invention, in a general and overall sense, relates to a nucleic acid-based system for simultaneously detecting and/or screening for two pathogens, such as Giardia and Cryptosporidium, in a single multiplex PCR assay format. While any variety of infectious pathogens may be detected employing the herein described methods, particular application of the present methods may be employed with Giardia and Cryptosporidium.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### Example 1

### Product for Pathogen Detection

The present example is directed to a description of the product as it exists in the format of different modules, the specific modules depending on the end use of the test and/or the PCR platform being used. For example, in some embodiments, 3 modules will be included.

These modules will include:
1. A specimen collection device:
2. DNA extraction reagents and consumables, and/or
3. PCR detection reagents and protocol

The specimen collection device will vary, depending upon the starting material (i.e., stool, water, soil, etc.). Likewise, the DNA extraction reagents will vary depending upon the starting material to provide optimized extractions for each type of starting material. The PCR detection reagents and protocol will also vary depending upon the starting material and/or the PCR platform used for the assay, providing optimized reagents and protocol for at least, for example, 4 major PCR platforms.

Production of Giardia/Crypto EZ-Amp™. The product may incorporate:

1. Sensitive DNA extraction methodology with reagents and magnetic beads customized specifically for the end use. And/or:

2. Sensitive, optimized PCR reagents with internal controls usable on Roche LightCycler, Cepheid SmartCycler, ABI 7300/7500, Corbett Roto gene, Finnzyme qPCR platform, and/or BioRad iCycler.

### Example 2

### Flowchart for Optimizing the Multiplex Assay onto SmartCycler

The present example demonstrates the utility of the present invention for providing a simultaneous PCR detection assay. In this particular example, the Roche LightCycler assay platform is used. This flow chart may be modified and applied for optimizing this product on the ABI7300.7500, Cepheid SmartCycler, Corbett Roto gene, Finnzyme qPCR platform, and/or BioRad iCycler PCR platforms.

Optimizing multiplex assays included several tasks. First singleplex PCR is performed with the primers, amplification confirmed by gel electrophoresis, and then multiplex PCR performed by adding all primers. Because multiplex PCR involves multiple templates that competitively co-amplify, biased amplification can occur. If sensitivity is diminished in multiplex vs. singleplex, as measured by increase in multiplex C_{T}, the following multiplex PCR variables are to be addressed sequentially.

1. Adjust primer concentration. Sensitivity of PCR amplification depends on the primer-to-template ratio. Too high a primer-to-template ratio decreases sensitivity because primer-dimerization is favored (at approximately 0.5 μM). Sensitivity can decrease if primer:template ratio is too low because product will not accumulate exponentially. On the other hand, lowering primer concentration has improved amplification by minimizing primer dimmers by the Genaco system [4]. In the end, primers are first added in equimolar amounts and then must be adjusted empirically up and down in a large matrix [21].

2. Increase MgCI₂/dNTP ratio. Adjusting MgCI₂ concentration may improve multiplex PCR amplification, presumably because Taq DNA polymerase activity is dependent on free [Mg⁺⁺] (and free Mg⁺⁺ is bound by dNTP). Increasing MgCI₂ concentration to 4 or 8 mM improves threshold for amplification in multiplex qPCR. dNTP stocks are sensitive to freeze-thaw cycles and should be aliqoted into small amounts, an effect not problematic with singleplex PCR.

3. Cycling conditions.
a. Annealing conditions. Lower annealing temperatures often increase multiplex PCR efficiency when DNA template is limiting. For the present multiplex Giardia qPCR assay, an optimal multiplex temperature of 54° C. was identified, though the primers were optimal at 60° C. in singleplex.
b. Extension. It has been reported that yield is increased for 100-300 bp amplicons by decreasing extension temperature (e.g., from 72° C. to 65° C.[21]). Additionally, increasing extension time (from 1 min to 4 min) produced visibly higher yields [21].
c. Denaturation. Although differential denaturation can occur with short AT-rich vs. long GC-rich sequences and responds to increases in denaturation temperature, denaturation duration, and salt concentrations, this should not be an issue with our amplicons of similar size.

4. PCR adjuvants. The usefulness of PCR adjuvants (DMSO, glycerol, betaine, and BSA) can be considered empirically for a multiplex reaction. A 6% DMSO and 2 μg/μl BSA was identified as beneficial for the Giardia qPCR assay. Acetylated BSA in high concentrations could inhibit the PCR [33] so proteinase-free BSA fractions are used. Such additives may act by preventing stalling of DNA polymerization or as stabilizing agents.

### Example 3

### Internal Control Construct (ICC)

A common problem of PCR, particularly in stool, is failure of DNA amplification due to the presence of inhibitory substances in samples. PCR-inhibitory substances are especially present in stool samples. The International Standard Organization has proposed a general guideline for PCR testing that requires the presence of an internal control in each PCR reaction [24]. Thus, if a PCR assay is to be validated through a multi-center collaborative trial it must contain an internal control.

There are two distinct mechanisms for false negative PCR: Low-yield DNA extraction from stool, or inhibition of PCR amplification. Poor DNA extraction from stool can occur due to incomplete cell lysis, DNA degradation in stool, or inefficient binding to the purification template. PCR inhibition can occur through carryover of inhibitory compounds such as mucoglycoproteins and proteases from stool. One investigator studied 78 stool specimens using a single DNA extraction procedure and PCR assay [3]; 9% of specimens exhibited extraction failure and 13% exhibited PCR inhibition. In other words there is specimen-to-specimen variability in either mechanism.

A known DNA template will be generated that can be detected with the same primer set used to detect Cryptosporidium, and a single probe specific for this synthetic DNA template. To do so, this synthetic DNA, along with the single, specific probe, will be synthesized. The synthetic DNA sequence will be transformed into a generic plasmid (such as pJ201) and then transfected into E. coli for cloning. This internal control can be used in one of two ways:

1.) E. coli transformants containing the internal control sequence plasmid will be used to spike a stool specimen prior to DNA extraction, and then the spiked stool specimen will be processed as the internal control through the entire DNA extraction and PCR procedure, or

2.) purified, extracted internal control plasmid will be spiked into PCR reagents (include all PCR reagents, Cryptosporidium and Giardia primers and probes, and Internal Control primers and probes) prior to PCR amplification, and then spiked PCR reagents will be amplified and detected by PCR procedure.

Detection of the 250 bp internal control fragment by qPCR will be required for interpretation of a given specimen, and will indicate either sufficient DNA extraction or proper PCR amplification, depending upon the point at which the internal control is added to the process.

Sequences for the internal control—Sequences for the internal control for multiplex detection of Cryptosporidium and Giardia are as follows. Additional internal control sequences will be generated for other PCR tests based on similar sequences to those below:

Internal Control Construct Sequence with Primer sequences (1 and 2):

(SEQ ID NO: 7)

5′- GCC TAC CGT GGC AAT GAA GTT AGT AGT GCG ATC
CTT TCT GAC TTT TGT CGT GCT GTG ACG GTG CTT GCC
ATG CGA ACA GCT GCA CAG GTA CTC GAG GGA AGG CAC
GTA AAT TTA GTC CCC CAA TAA ATA ACA GGC CGC TGT
TGA GCA CAA GCA GCT AGC GCC GTT TTA GCC ACA TGT
ACC CAG TAT ATA TGT CAC GAG AGG ATA GGC GAA TTG
GAA TGG TCA GGC CGA CAA GAA ATA ACA ATA CAG GAC
TTT -3′

Internal Control Primer Sequences: Identical to Cryptosporidium primer sequences.

Forward Primer:
Crypto Forward (primer 1)

(SEQ ID NO: 2)

5′- GCC TAC CGT GGC AAT GA-3′
Reverse Primer:
Crypto Reverse (primer 2)

(SEQ ID NO: 4)

5′- AAA GTC CTG TAT TGT TAT TTC TTG TC-3′

Internal Control Detection Probe Sequences:

(SEQ ID NO: 6)

Donor Probe:
5′- GT CGT GCT GTG ACG GTG CTT GCC ATG CGA A -3′
Acceptor Probe:
5′- A GCT GCA CAG GTA CTC GAG GGA AGG CAC GT -3′

Detection of this Internal Control (“IC”) plasmid will involve adding the forward and reverse primers for the IC (same primer set as those for amplifying Cryptosporidium) to each PCR reaction, as well as unique IC hybridization FRET probes that will specifically bind to the IC 250 bp synthetic fragment. In some embodiments, the donor probe will include a green fluorophore modification at its 3′ end (FAM, FITC, Alexa flour 488, or other complimentary fluorophore) and the acceptor probe will include a red fluorophore modification at its 5′ end (LC 705, Texas Red, or other high-mage red fluorophore), which will detect in channel 3 of the LightCycler 1.5/2.0. A FRET reaction is necessary in this case due to the low excitation (470 nm) and emission (540 nm) of FAM. This IC system will also work without modifications in the SmartCycler assay, as well as subsequent assays developed for various PCR platforms (ABI 7300/7500, Corbett Roto gene, Finnzyme qPCR platform, BioRad iCycler, etc. The same IC sequences and single-labeled hybridization probe will be employed with 3′-fluorophore modification for all qPCR platforms. One issue, among others, that has been solved in the present IC design is the difficulties associated with the addition of too many primers and probes in a single, multiplex reaction. Specifically, reduced PCR efficiencies may be experienced with increasing numbers of primers/probes to an individual reaction. In order to control for this particular problem, and by way of example only, the present methods and assays may include 2 Giardia primers, 2 Giardia probes, 2 Cryptosporidium primers (that also amplify the IC), 2 Cryptosporidium probes, and 2 IC probes (10 oligos/probes per reaction)(IC is Internal Control).

The internal control is detected at the same wavelength as the Giardia template, and is loaded at levels barely detectable to avoid competition for reagents with either Cryptosporidium or Giardia. In the event that both Giardia and the IC are detected in the same sample, they are discriminated by melt curve analysis (looking at the temperature at which the probes disassociate with the template). The temperature difference between Giardia and IC probes will be in the range of 5-10 degrees Celsius.

### Internal Control Construct (ICC):

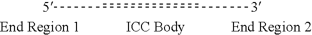

In some embodiments, the ICC structure comprises an ICC body, an end region 1 and an end region 2. The end region 1 and the end region 2 may comprise the same or different base pair sequences. The end region 1 and end region 2 may in some embodiments comprise a sequence that corresponds to the base pair sequence of a primer sequence of a target microorganism to be detected according to the PCR techniques described herein.

In some embodiments, of the above ICC structure, the ICC Body region may be described as having a length of about 190 to about 210 base pairs (bp). In some embodiments, the ICC body may be described as having a length of 207 bp. In one particular embodiment of the ICC, the ICC body will comprise a sequence as defined by the following 207 bp sequence:

SEQ ID NO: 1:
5′- GAA GTT AGT AGT GCG ATC CTT TCT GAC TTT TGT
CGT GCT GTG ACG GTG CTT GCC ATG CGA ACA GCT GCA
CAG GTA CTC GAG GGA AGG CAC GTA AAT TTA GTC CCC
CAA TAA ATA ACA GGC CGC TGT TGA GCA CAA GCA GCT
AGC GCC GTT TTA GCC ACA TGT ACC CAG TAT ATA TGT
CAC GAG AGG ATA GGC GAA TTG GAA TGG TCA GGC C -3′

In some embodiments of the ICC structure, the end region 1 is described as a sequence located at the 5′ end of the structure. The end region one may be further described as a sequence comprising 15 base pairs (bp) to 30 base pairs (bp). In some embodiments, the end region 1 is a sequence of 17 bp. Solely for purposes of example, the end region 1 may comprise a sequence of a primer sequence as described herein as a forward primer for Cryptosporidium. In some embodiments, the specific sequence of the end region 1 having a length of 17 bp is

| | |
|---|---|
| Crypto Forward (primer 1) | |
| 5′- GCC TAC CGT GGC AAT GA-3′ | (SEQ ID NO: 2) |

By way of further example, the end region 1 may comprise a sequence of a primer sequence as described herein as a forward primer for Giardia. In these embodiments, the specific sequence of the end region 1 that corresponds to a forward primer for Giardia posses a length of 17 bp, and has a sequence of

| | |
|---|---|
| 5′-GGA CGG CTC AGG ACA AC-3′ | (SEQ ID NO: 3) |

In some embodiments of the ICC structure, the end region 2 is described as a sequence located at the 3′ end of the structure. The end region two (2) may be further described as a sequence comprising 15 base pairs (bp) to 30 base pairs (bp). In some embodiments, the end region 2 is a sequence of 26 bp. Solely for purposes of example, the end region 2 may comprise a sequence of a primer sequence as described herein as a reverse primer for Cryptosporidium In some embodiments, the specific sequence of the end region 2 having a length of 26 bp is

Crypto Reverse (primer 2)

(SEQ ID NO: 4)

5′-AAA GTC CTG TAT TGT TAT TTC TTG TC-3′

By way of further example, the end region 2 may comprise a sequence of a primer sequence as described herein as a reverse primer for Giardia. In these embodiments, the specific sequence of the end region 2 that corresponds to a reverse primer for Giardia posses a length of 19 bp, and has a sequence of

| | |
|---|---|
| 5′-GGA GTC GAA CCC TGA TTCT-3′ | (SEQ ID NO: 5) |

### Example 4

### DNA Capture and Extraction Method

Any PCR assay used by a clinical laboratory needs to be wed to a rapid and easy DNA extraction method in order to gain traction against traditional methods. The primary manual nucleic acid extraction kits used by clinical laboratories are the Qiagen QIAamp kits. The present studies demonstrated here establish good results using these products. Therefore, the Crypto/Giardia EZ-Amp™ kit may utilize a Qiagen-based DNA extraction methodology. However, certain steps may be and have been modified in order to increase sensitivity of detection and speed the protocol. Other embodiments may use other DNA extraction methodologies currently used in clinical, veterinarian, and/or water testing laboratories, particularly automated DNA extraction methods.

### Example 5

### PCR vs. Merifluor

The PCR test was compared to the Merifluor Cryptosporidium/Giardia® IFA test. The Merifluor assay is widely used in clinical laboratories and often considered a gold-standard test more sensitive than other antigen detection kits [15, 29]. For example, versus the Merifluor IFA, the sensitivity of EIA for Giardia ranged from 94% to 99% and the sensitivity of EIA for Cryptosporidium ranged from 98% to 99%; specificities were 100% [14]. Merifluor uses FITC-labeled antibodies specific for Cryptosporidium and Giardia that bind to the surface of the parasites. Upon fluorescent microscopy the two parasites are distinguished by visual comparison and size. Background material and/or other organisms are counterstained red.

The Crypto/Giardia test will exhibit greater sensitivity than the Merifluor. This advantage makes the assay improved over PCR-based techniques. The PCR will be run for 45 cycles. Using the example shown in the table, the following will be calculated:

Sensitivity of PCR vs. IFA=TP/(TP+FN)=50/(50+10)=94%

Specificity of PCR vs. IFA=FP/(FP+TN)=195/(195+50)=91%

The presently disclosed capture/amplification technique is more sensitive than antigen detection. Comparison will be made of the PCR C_{T} between the “TP” and “FP” specimens, as finding a correlation between high DNA load (low C_{T} and microscopic positivity (“TP”) would be even further validating.

Any discrepant data will be re-assayed with an additional PCR that amplifies a Cryptosporidium and Giardia non-18S gene. Namely, PCR assays for the Cryptosporidium oocyst wall protein (COWP 702, 151-bp) and Giardia β-giardin (β-giardin P241, 74-bp) will be run [17]. These are SYBR-green based qPCR assays. [1]. The gold-standard will then be identified for discrepant data as the result obtained from 2 out of 3 tests. If, for instance, the second PCR is positive for 16 of the 20 “FP” results (and negative for 4) and is positive for 2 of the 5 “FN” results (and negative for 3), sensitivity/specificity would be re-calculated as follows:

| | Gold-standard+ | Gold-standard− |
|---|---|---|
| New test (PCR)+ | TP (ex, 80 + 16 = 96) | FP (was 20, move 16 |
| | | to TP, now = 4) |
| New test (PCR) | FN (was 5, move 3 | TN (ex, 195 + 3) |
| | to TN, now = 2) | |

Therefore, sensitivity of PCR vs. gold-standard=TP/(TP+FN)=96/(96+2)=98%
Specificity of PCR vs. gold-standard=FP/(FP+TN)=198/(198+4)=98%

### Example 6

The present example demonstrates the utility of the present invention for use in testing a sample for contaminants, such as in the testing of municipal water supplies for contaminants. The present methods present an easier and less-expensive test for testing water supplies and water environments for contaminants.

100 water samples will be tested from diverse areas in Bangkok, including the wastewater canals (high burden), water purification center (low burden or negative), and ozonated bottled water (should negative). All water samples will be tested using the comparator EPA 1622/1623. Results from 50 positive and 50 negative will be collected via EPA 1622/1623. The qPCR assay that will be utilized will be the LightCycler assay, since this 96-well format will be convenient to high-volume water testing laboratories (speed of the LightCycler is less of an advantage). Similar to the KCMC plan, discrepant results will be resolved by comparing quantitative C_{T} values in the EPA 1622/1623 positive (presumably low C_{T}) versus negative (presumably high C_{T}) groups. The same tiebreaker approach with a third PCR assay will be utilized.

Focus will be on detection. However the potential exists to incorporate molecular methods to determine not only presence but viability of protozoal cysts. Molecular methods, such as RT-PCR [19, 27, 47], may therefore be included in some embodiments of the methods as a complementary screen to assess viability. RT-PCR may complicate the present capture and PCR detection method which is optimized for DNA, and would require and additional set of primers for reverse transcription of cDNA prior to PCR. By way of example, it is envisioned that the present technique may employ a sample, such as a water sample, that has been treated with DNAse, thus promoting the disruption of any cysts that may be present in a water sample. It is envisioned that the DNAse will penetrate non-viable and disrupted cysts. Alternatively, a sample may be treated with ethidium monoazide (EMA), which also will penetrate non-viable dead cells and covalently bind to DNA such that it cannot be PCR amplified [43]. EMA has been used in several similar applications, such as determining the viability of Campylobacter in environmental sources [43]. Both the DNAse and EMA water-treatment approaches will be titrated and compared with EPA 1622/1623's standard viability criteria of propidium iodide and DAPI exclusion. However, this will occur after optimization of the PCR detection has been accomplished for detection. Stool specimens will be collected in Tanzania and water data in Bangkok.

### Example 7

The present example is provided to demonstrate the protocol to be used in the analysis of a specimen suspected to be infected or to contain two (2) or more environmental pathogens, such as Cryptosporidium and Giardia.

Cryptosporidium/Giardia qPCR Protocol (LightCycler-Roche):

The following presents the step-by-step method by which the diagnostic test of a sample of interest will be run.

1. Setup LightCycler

Turn on thermocycler

Boot up computer and load LightCycler software; select “run” from front screen

Click “OK” when asked to run diagnostic

Load or create experiment file

2. Setup PCR Reactions

All reagents should always be kept on ice; hybprobe reagents should not be frozen after combining; probes should be protected from light at all times; avoid freeze-thaw of all reagents.

Thaw and prepare reagents according to kit instructions

Master mix supply: Make a master mix with the following components:

| | Final | μl per |
|---|---|---|
| Component | concentration | reaction |
| Water | | 1.5 |
| MgCl | 6 mM | 4 |
| DMSO | 8% of final | 1.5 |
| | volume | |
| C Primer 1 | 0.6 μM | 1 |
| C Primer 2 | 0.6 μM | 1 |
| G Primer 1 | 0.6 μM | 1 |
| G Primer 2 | 0.6 μM | 1 |
| C Probe 1 | 0.2 μM | 1 |
| C Probe 2 | 0.2 μM | 1 |
| G Probe 1 | 0.2 μM | 1 |
| G Probe 2 | 0.2 μM | 1 |
| HybProbe Buffer | | 2 |
| Template | | 3 |

Negative (No Template) Control

a. Pipette 17 ul of the master mix into each glass capillary and then add 3 ul template to each

b. Cap glass capillaries and then centrifuge on slow speed for 10 seconds (using centrifuge adaptors)

c. Remove capillaries from centrifuge and load into LightCycler carousel, dropping capillaries into the spaces to avoid breakage

d. Press each capillary down into the carousel and then load the carousel into the LightCycler and close lid.

Run PCR Reactions

a. Settings are correct for the PCR protocol, and data collection is turned on at the appropriate steps; run conditions as follows:

| Step | Temperature (C.) | Time | Acquisition Mode |
|---|---|---|---|
| Hot Start | 95 | 15 min | None |

Amplification

| | | | |
|---|---|---|---|
| Denature | 95 | 10 sec | None |
| Anneal | 50 | 20 sec | Single |
| Extend | 72 | 30 sec | None |

Melting Curve

| | | | |
|---|---|---|---|
| Denature | 95 | 0 sec | None |
| Anneal | 45 | 30 sec | None |
| Melting | 95 (slope = 0.1 C./sec | 0 sec | Continuous |

a. Save the study/run

b. Click “run” to start the study/run

c. Enter the number of samples and then label them as appropriate in ‘edit samples.

Analyze Data

a. The data analysis module will open automatically at the end of the run

b. Select “CCC” color compensation file

c. View the quantification and melt curve data sections (toggle the button on the top left of the screen to move between quantification and melt); print the appropriate reports and/or save images

Materials/Primers/Probes/Reagents

a. Primers

i. Giardia Forward (primer 1)

(SEQ ID NO: 3)

5′-GGA CGG CTC AGG ACA AC-3′
ii. Giardia Reverse (primer 2)

(SEQ ID NO: 5)

5′-GGA GTC GAA CCC TGA TTC T-3′.
iii. Crypto Forward (primer 1)

(SEQ ID NO: 2)

5′-GCC TAC CGT GGC AAT GA-3′
iv. Crypto Reverse (primer 2)

(SEQ ID NO: 4)

5′-AAA GTC CTG TAT TGT TAT TTC TTG TC-3′

b. Probes

i. Giardia Probe 1

(SEQ ID NO: 8)

5′-CGT GAC GCA GCG ACG G-Fluorescein-3′
ii. Giardia Probe 2

(SEQ ID NO: 9)

5′-LCRed705-CGC CCG GGC TTC CGG-Phosphate-3′
iii. Crypto Probe 1

(SEQ ID NO: 10)

5′-CGG CTA CCA CAT CTA AGG AAG GC-Fluorescein-3′
iv. Crypto Probe 2

(SEQ ID NO: 11)

5′-LCRed640-CAG GCG CGC AAA TTA CCC AAT CCT A-
Phosphate-3′

d. Reagents
1. LightCycler FastStart DNA Master HybProbe (Cat. No. 03 003 248 001)
2. Color Compensation Set (Cat. No. 12 158 850 001) used to subtract F2 crossover from F3 channel

### BIBLIOGRAPHY

The following articles/journals/patents and other reference materials are specifically incorporated herein by reference.
Haque, R., S. Roy, et al. (2007). “Multiplex real-time PCR assay for detection of Entamoeba histolytica, Giardia intestinalis, and Cryptosporidium spp.” Am J Trop Med Hyg 76(4): 713-7.
Ng, C. T., C. A. Gilchrist, et al. (2005). “Multiplex real-time PCR assay using Scorpion probes and DNA capture for genotype-specific detection of Giardia lamblia on fecal samples.” J Clin Microbiol 43(3): 1256-60.
Stroup, S. E., S. Roy, et al. (2006). “Real-time PCR detection and speciation of Cryptosporidium infection using Scorpion probes.” J Med Microbiol 55(Pt 9): 1217-22.
Abe, N. and M. Iseki (2004). “Identification of Cryptosporidium isolates from cockatiels by direct sequencing of the PCR-amplified small subunit ribosomal RNA gene.” Parasitol Res 92(6): 523-6.
Abe, N., I. Kimata, et al. (2002). “Comparative study of PCR-based Cryptosporidium discriminating techniques with a review of the literature.” Kansenshogaku Zasshi 76(10): 869-81.
Abe, N., I. Kimata, et al. (2003). “Identification of genotypes of Giardia intestinalis isolates from dogs in Japan by direct sequencing of the PCR amplified glutamate dehydrogenase gene.” Vet Med Sci 65(1): 29-33.
Alves, M., O. Matos, et al. (2001). “Multilocus PCR-RFLP analysis of Cryptosporidium isolates from HIV-infected patients from Portugal.” Ann Trop Med Parasitol 95(6): 627-32.
Alves, M., O. Matos, et al. (2000). “PCR-RFLP analysis of Cryptosporidium parvum isolates from HIV-infected patients in Lisbon, Portugal.” Ann Trop Med Parasitol 94(3): 291-7.
Amar, C. F., P. H. Dear, et al. (2003). “Detection and genotyping by real-time PCR/RFLP analyses of Giardia duodenalis from human faeces.” J Med Microbiol 52(Pt 8): 681-3.
Amar, C. F., P. H. Dear, et al. (2004). “Detection and identification by real time PCR/RFLP analyses of Cryptosporidium species from human faeces.” Lett Appl Microbiol 38(3): 217-22.
Amar, C. F., P. H. Dear, et al. (2002). “Sensitive PCR-restriction fragment length polymorphism assay for detection and genotyping of Giardia duodenalis in human feces.” J Clin Microbiol 40(2): 446-52.
Anceno, A. J., H. Katayama, et al. (2007). “IMS-free DNA extraction for the PCR-based quantification of Cryptosporidium parvum and Giardia lamblia in surface and waste water.” Int J Environ Health Res 17(4): 297-310.
Anceno, A. J., H. Katayama, et al. (2007). “IMS-free DNA extraction for the PCR-based quantification of Cryptosporidium parvum and Giardia lamblia in surface and waste water.” Int J Environ Health Res 17(4): 297-310.
Awad-el-Kariem, F. M., D. C. Warhurst, et al. (1994). “Detection and species identification of Cryptosporidium oocysts using a system based on PCR and endonuclease restriction.” Parasitology 109 (Pt 1): 19-22.
Balatbat, A. B., G. W. Jordan, et al. (1996). “Detection of Cryptosporidium parvum DNA in human feces by nested PCR.” J Clin Microbiol 34(7): 1769-72.
Bertrand, I., L. Albertini, et al. (2005). “Comparison of two target genes for detection and genotyping of Giardia lamblia in human feces by PCR and PCR-restriction fragment length polymorphism.” J Clin Microbiol 43(12): 5940-4.
Bertrand, I., C. Gantzer, et al. (2004). “Improved specificity for Giardia lamblia cyst quantification in wastewater by development of a real-time PCR method.” J Microbiol Methods 57(1): 41-53.
Bialek, R., N. Binder, et al. (2002). “Comparison of fluorescence, antigen and PCR assays to detect Cryptosporidium parvum in fecal specimens.” Diagn Microbiol Infect Dis 43(4): 283-8.
Bienz, M., M. Siles-Lucas, et al. (2001). “Use of a novel DNA melting profile assay for the identification of PCR-amplified genomic sequences encoding for variant-specific surface proteins from the clonal GS/M-83-H7 line of Giardia lamblia.” Parasitol Res 87(12): 1011-5.
Cai, J., M. D. Collins, et al. (1992). “PCR cloning and nucleotide sequence determination of the 18S rRNA genes and internal transcribed spacer 1 of the protozoan parasites Cryptosporidium parvum and Cryptosporidium muris.” Biochim Biophys Acta 1131(3): 317-20.
Cai, X., K. M. Woods, et al. (2005). “Application of quantitative real-time reverse transcription-PCR in assessing drug efficacy against the intracellular pathogen Cryptosporidium parvum in vitro.” Antimicrob Agents Chemother 49(11): 4437-42.
Carey, C. M., H. Lee, et al. (2006). “Comparison of most probable number-PCR and most probable number-foci detection method for quantifying infectious Cryptosporidium parvum oocysts in environmental samples.” J Microbiol Methods 67(2): 363-72.
Champliaud, D., P. Gobet, et al. (1998). “Failure to differentiate Cryptosporidium parvum from C. meleagridis based on PCR amplification of eight DNA sequences.” Appl Environ Microbiol 64(4): 1454-8.
Chung, E., J. E. Aldom, et al. (1999). “PCR-based quantitation of Cryptosporidium parvum in municipal water samples.” J Microbiol Methods 38(1-2): 119-30.
Coupe, S., C. Sarfati, et al. (2005). “Detection of Cryptosporidium and identification to the species level by nested PCR and restriction fragment length polymorphism.” J Clin Microbiol 43(3): 1017-23.
Deng, M. Q., D. O. Cliver, et al. (1997). “Immunomagnetic capture PCR to detect viable Cryptosporidium parvum oocysts from environmental samples.” Appl Environ Microbiol 63(8): 3134-8.
Di Giovanni, G. D., F. H. Hashemi, et al. (1999). “Detection of infectious Cryptosporidium parvum oocysts in surface and filter backwash water samples by immunomagnetic separation and integrated cell culture-PCR.” Appl Environ Microbiol 65(8): 3427-32.
Di Giovanni, G. D. and M. W. LeChevallier (2005). “Quantitative-PCR assessment of Cryptosporidium parvum cell culture infection.” Appl Environ Microbiol 71(3): 1495-500.
Filkorn, R., A. Wiedenmann, et al. (1994). “Selective detection of viable Cryptosporidium oocysts by PCR.” Zentralbl Hyg Umweltmed 195(5-6): 489-94.
Filkorn-Kaiser, R., K. Botzenhart, et al. (2005). “Development and test for long-term stability of a synthetic standard for a quantitative Cryptosporidium parvum LightCycler PCR assay.” J Water Health 3(1): 15-25.
Fischer, P., H. Taraschewski, et al. (1998). “Detection of Cryptosporidium parvum in human feces by PCR.” Tokai J Exp Clin Med 23(6): 309-11.
Fontaine, M. and E. Guillot (2002). “Development of a TaqMan quantitative PCR assay specific for Cryptosporidium parvum.” FEMS Microbiol Lett 214(1): 13-7.
Fontaine, M. and E. Guillot (2003). “An immunomagnetic separation-real-time PCR method for quantification of Cryptosporidium parvum in water samples.” J Microbiol Methods 54(1): 29-36.
Fontaine, M. and E. Guillot (2003). “Study of 18S rRNA and rDNA stability by real-time RT-PCR in heat-inactivated Cryptosporidium parvum oocysts.” FEMS Microbiol Lett 226(2): 237-43.
Garces, G., M. Effenberger, et al. (2006). “Quantification of Cryptosporidium parvum in anaerobic digesters treating manure by (reverse-transcription) quantitative real-time PCR, infectivity and excystation tests.” Water Sci Technol 53(8): 195-202.
Ghosh, S., A. Debnath, et al. (2000). “PCR detection of Giardia lamblia in stool: targeting intergenic spacer region of multicopy rRNA gene.” Mol Cell Probes 14(3): 181-9.
Gibbons, C. L. and F. M. Awad-El-Kariem (1999). “Nested PCR for the detection of Cryptosporidium parvum.” Parasitol Today 15(8): 345.
Gile, M., D. C. Warhurst, et al. (2002). “A multiplex allele specific polymerase chain reaction (MAS-PCR) on the dihydrofolate reductase gene for the detection of Cryptosporidium parvum genotypes 1 and 2.” Parasitology 125(Pt 1): 35-44.
Gobet, P., J. C. Buisson, et al. (1997). “Detection of Cryptosporidium parvum DNA in formed human feces by a sensitive PCR-based assay including uracil-N-glycosylase inactivation.” J Clin Microbiol 35(1): 254-6.
Gobet, P. and S. Toze (2001). “Sensitive genotyping of Cryptosporidium parvum by PCR-RFLP analysis of the 70-kilodalton heat shock protein (HSP70) gene.” FEMS Microbiol Lett 200(1): 37-41.
Godiwala, N. T., A. Vandewalle, et al. (2006). “Quantification of in vitro and in vivo Cryptosporidium parvum infection by using real-time PCR.” Appl Environ Microbiol 72(6): 4484-8.
Gomez-Couso, H., F. Freire-Santos, et al. (2004). “Detection of Cryptosporidium and Giardia in molluscan shellfish by multiplexed nested-PCR.” Int J Food Microbiol 91(3): 279-88.
Gomez-Couso, H., F. Freire-Santos, et al. (2004). “Detection of Cryptosporidium and Giardia in molluscan shellfish by multiplexed nested-PCR.” Int J Food Microbiol 91(3): 279-88.
Gomez-Couso, H., F. Mendez-Hermida, et al. (2006). “Levels of detection of Cryptosporidium oocysts in mussels (Mytilus galloprovincialis) by IFA and PCR methods.” Vet Parasitol 141(1-2): 60-5.
Guy, R. A., P. Payment, et al. (2003). “Real-time PCR for quantification of Giardia and Cryptosporidium in environmental water samples and sewage.” Appl Environ Microbiol 69(9): 5178-85.
Guy, R. A., P. Payment, et al. (2003). “Real-time PCR for quantification of Giardia and Cryptosporidium in environmental water samples and sewage.” Appl Environ Microbiol 69(9): 5178-85.
Guy, R. A., C. Xiao, et al. (2004). “Real-time PCR assay for detection and genotype differentiation of Giardia lamblia in stool specimens.” J Clin Microbiol 42(7): 3317-20.
Guyot, K., A. Follet-Dumoulin, et al. (2002). “PCR-restriction fragment length polymorphism analysis of a diagnostic 452-base-pair DNA fragment discriminates between Cryptosporidium parvum and C. meleagridis and between C. parvum isolates of human and animal origin.” Appl Environ Microbiol 68(4): 2071-6.
Hallier-Soulier, S. and E. Guillot (2003). “An immunomagnetic separation-reverse transcription polymerase chain reaction (IMS-RT-PCR) test for sensitive and rapid detection of viable waterborne Cryptosporidium parvum.” Environ Microbiol 5(7): 592-8.
Haque, R., S. Roy, et al. (2007). “Multiplex real-time PCR assay for detection of Entamoeba histolytica, Giardia intestinalis, and Cryptosporidium spp.” Am J Trop Med Hyg 76(4): 713-7.
Haque, R., S. Roy, et al. (2007). “Multiplex real-time PCR assay for detection of Entamoeba histolytica, Giardia intestinalis, and Cryptosporidium spp.” Am J Trop Med Hyg 76(4): 713-7.
Hashimoto, A., H. Sugimoto, et al. (2006). “Genotyping of single Cryptosporidium oocysts in sewage by semi-nested PCR and direct sequencing.” Water Res 40(13): 2527-32.
Higgins, J. A., R. Fayer, et al. (2001). “Real-time PCR for the detection of Cryptosporidium parvum.” J Microbiol Methods 47(3): 323-37.
Higgins, J. A., M. C. Jenkins, et al. (2001). “Rapid extraction of DNA From Escherichia coli and Cryptosporidium parvum for use in PCR.” Appl Environ Microbiol 67(11): 5321-4.
Hopkins, R. M., C. C. Constantine, et al. (1999). “PCR-based DNA fingerprinting of Giardia duodenalis isolates using the intergenic rDNA spacer.” Parasitology 118 (Pt 6): 531-9.
Jenkins, M. C., J. Trout, et al. (2000). “Estimating viability of Cryptosporidium parvum oocysts using reverse transcriptase-polymerase chain reaction (RT-PCR) directed at mRNA encoding amyloglucosidase.” J Microbiol Methods 43(2): 97-106.
Jiang, J., K. A. Alderisio, et al. (2005). “Development of procedures for direct extraction of Cryptosporidium DNA from water concentrates and for relief of PCR inhibitors.” Appl Environ Microbiol 71(3): 1135-41.
Johnson, D. W., N. J. Pieniazek, et al. (1995). “Development of a PCR protocol for sensitive detection of Cryptosporidium oocysts in water samples.” Appl Environ Microbiol 61(11): 3849-55.
Johnson, M. L., D. I. Berryman, et al. (2003). “A fluorescent based PCR assay for the detection and quantitation of Giardia duodenalis genotypes in mixed populations.” Infect Genet Evol 3(2): 97-102.
Karasudani, T., S. Aoki, et al. (2001). “Sensitive detection of Cryptosporidium oocysts in environmental water samples by reverse transcription-PCR.” Jpn J Infect Dis 54(3): 122-4.
Kato, S., G. Lindergard, et al. (2003). “Utility of the Cryptosporidium oocyst wall protein (COWP) gene in a nested PCR approach for detection infection in cattle.” Vet Parasitol 111(2-3): 153-9.
Kaucner, C. and T. Stinear (1998). “Sensitive and Rapid Detection of Viable Giardia Cysts and Cryptosporidium parvum Oocysts in Large-Volume Water Samples with Wound Fiberglass Cartridge Filters and Reverse Transcription-PCR.” Appl Environ Microbiol 64(11): 4627.
Keegan, A. R., S. Fanok, et al. (2003). “Cell culture-Taqman PCR assay for evaluation of Cryptosporidium parvum disinfection.” Appl Environ Microbiol 69(5): 2505-11.
Keino, M., H. Kumada, et al. (2006). “Trial of nested PCR by using DNA templates extracted from preparations for microscopic tests for Cryptosporidium oocysts and Giardia cysts.” Jpn J Infect Dis 59(4): 278-9.
Laberge, I., A. Ibrahim, et al. (1996). “Detection of Cryptosporidium parvum in raw milk by PCR and oligonucleotide probe hybridization.” Appl Environ Microbiol 62(9): 3259-64.
Latifah, I., K. Y. Teoh, et al. (2005). “Detection of giardine gene in local isolates of Giardia duodenalis by polymerase chain reaction (PCR).” Malays J Pathol 27(2): 83-9.
LeChevallier, M. W., G. D. Di Giovanni, et al. (2003). “Comparison of method 1623 and cell culture-PCR for detection of Cryptosporidium spp. in source waters.” Appl Environ Microbiol 69(2): 971-9.
Lee, J. H., J. Lee, et al. (2006). “Detection and genotyping of Giardia intestinalis isolates using intergenic spacers (IGS)-based PCR.” Korean J Parasitol 44(4): 343-53.
Lee, K. and K. Kim (2003). “A colorimetric confirmation method for DNA amplification in PCR and its application to the detection of Giardia lamblia cysts.” Biotechnol Lett 25(20): 1739-42.
Leng, X., D. A. Mosier, et al. (1996). “Differentiation of Cryptosporidium parvum, C. muris, and C. baileyi by PCR-RFLP analysis of the 18S rRNA gene.” Vet Parasitol 62(1-2): 1-7.
Leng, X., D. A. Mosier, et al. (1996). “Simplified method for recovery and PCR detection of Cryptosporidium DNA from bovine feces.” Appl Environ Microbiol 62(2): 643-7.
Leoni, F., C. I. Gallimore, et al. (2003). “A rapid method for identifying diversity within PCR amplicons using a heteroduplex mobility assay and synthetic polynucleotides: application to characterisation of dsRNA elements associated with Cryptosporidium.” J Microbiol Methods 54(1): 95-103.
Limor, J. R., A. A. Lal, et al. (2002). “Detection and differentiation of Cryptosporidium parasites that are pathogenic for humans by real-time PCR.” J Clin Microbiol 40(7): 2335-8.
Lindergard, G., D. V. Nydam, et al. (2003). “The sensitivity of PCR detection of Cryptosporidium oocysts in fecal samples using two DNA extraction methods.” Mol Diagn 7(3-4): 147-53.
Lowery, C. J., J. E. Moore, et al. (2000). “Detection and speciation of Cryptosporidium spp. in environmental water samples by immunomagnetic separation, PCR and endonuclease restriction.” J Med Microbiol 49(9): 779-85.
Lowery, C. J., P. Nugent, et al. (2001). “PCR-IMS detection and molecular typing of Cryptosporidium parvum recovered from a recreational river source and an associated mussel (Mytilus edulis) bed in Northern Ireland.” Epidemiol Infect 127(3): 545-53.
MacDonald, L. M., K. Sargent, et al. (2002). “The development of a real-time quantitative-PCR method for characterisation of a Cryptosporidium parvum in vitro culturing system and assessment of drug efficacy.” Mol Biochem Parasitol 121(2): 279-82.
Mahbubani, M. H., F. W. Schaefer, 3rd, et al. (1998). “Detection of Giardia in environmental waters by immuno-PCR amplification methods.” Curr Microbiol 36(2): 107-13.
Masago, Y., K. Oguma, et al. (2006). “Quantification and genotyping of Cryptosporidium spp. in river water by quenching probe PCR and denaturing gradient gel electrophoresis.” Water Sci Technol 54(3): 119-26.
Mathis, A., P. Deplazes, et al. (1996). “[PCR for detection and characterization of parasites (Leishmania, Echinococcus, Microsporodia, Giardia)].” Schweiz Arch Tierheilkd 138(3): 133-8.
Mayer, C. L. and C. J. Palmer (1996). “Evaluation of PCR, nested PCR, and fluorescent antibodies for detection of Giardia and Cryptosporidium species in wastewater.” Appl Environ Microbiol 62(6): 2081-5.
McGlade, T. R., I. D. Robertson, et al. (2003). “High prevalence of Giardia detected in cats by PCR.” Vet Parasitol 110(3-4): 197-205.
Millar, C., J. Moore, et al. (2001). “Successful PCR amplification of genomic DNA from Cryptosporidium parvum oocysts extracted from a human faecal sample: a rapid and simple method suited for outbreak analysis.” Int J Hyg Environ Health 204(2-3): 191-4.
Miller, K. M. and C. R. Sterling (2007). “Sensitivity of Nested PCR Detection for Low Numbers of Giardia lamblia Cysts.” Appl Environ Microbiol.
Monis, P. T. and C. P. Saint (2001). “Development of a nested-PCR assay for the detection of Cryptosporidium parvum in finished water.” Water Res 35(7): 1641-8.
Morgan, U. M. and R. C. Andrew Thompson (1998). “PCR detection of Cryptosporidium-addendum.” Parasitol Today 14(11): 469-70.
Morgan, U. M., C. C. Constantine, et al. (1997). “Differentiation between human and animal isolates of Cryptosporidium parvum using rDNA sequencing and direct PCR analysis.” J Parasitol 83(5): 825-30.
Morgan, U. M., P. Deplazes, et al. (1999). “Sequence and PCR-RFLP analysis of the internal transcribed spacers of the rDNA repeat unit in isolates of Cryptosporidium from different hosts.” Parasitology 118 (Pt 1): 49-58.
Morgan, U. M., P. A. O'Brien, et al. (1996). “The development of diagnostic PCR primers for Cryptosporidium using RAPD-PCR.” Mol Biochem Parasitol 77(1): 103-8.
Morgan, U. M., L. Pallant, et al. (1998). “Comparison of PCR and microscopy for detection of Cryptosporidium parvum in human fecal specimens: clinical trial.” J Clin Microbiol 36(4): 995-8.
Morgan, U. M. and R. C. Thompson (1998). “PCR detection of Cryptosporidium: the way forward?” Parasitol Today 14(6): 241-5.
Nantavisai, K., M. Mungthin, et al. (2007). “Evaluation of the sensitivities of DNA extraction and PCR methods for detection of Giardia duodenalis in stool specimens.” J Clin Microbiol 45(2): 581-3.
Ng, C. T., C. A. Gilchrist, et al. (2005). “Multiplex real-time PCR assay using Scorpion probes and DNA capture for genotype-specific detection of Giardia lamblia on fecal samples.” J Clin Microbiol 43(3): 1256-60.
Nichols, R. A., B. M. Campbell, et al. (2003). “Identification of Cryptosporidium spp. oocysts in United Kingdom noncarbonated natural mineral waters and drinking waters by using a modified nested PCR-restriction fragment length polymorphism assay.” Appl Environ Microbiol 69(7): 4183-9.
Ochiai, Y., C. Takada, et al. (2005). “Detection and discrimination of Cryptosporidium parvum and C. hominis in water samples by immunomagnetic separation-PCR.” Appl Environ Microbiol 71(2): 898-903.
Patel, S., S. Pedraza-Diaz, et al. (1999). “The identification of Cryptosporidium species and Cryptosporidium parvum directly from whole faeces by analysis of a multiplex PCR of the 18S rRNA gene and by PCR/RFLP of the Cryptosporidium outer wall protein (COWP) gene.” Int J Parasitol 29(8): 1241-7.
Read, C. M., P. T. Monis, et al. (2004). “Discrimination of all genotypes of Giardia duodenalis at the glutamate dehydrogenase locus using PCR-RFLP.” Infect Genet Evol 4(2): 125-30.
Reed, C., G. D. Sturbaum, et al. (2002). “Cryptosporidium parvum mixed genotypes detected by PCR-restriction fragment length polymorphism analysis.” Appl Environ Microbiol 68(1): 427-9.
Rimhanen-Finne, R., A. Horman, et al. (2002). “An IC-PCR method for detection of Cryptosporidium and Giardia in natural surface waters in Finland.” J Microbiol Methods 50(3): 299-303.
Rimhanen-Finne, R., A. Horman, et al. (2002). “An IC-PCR method for detection of Cryptosporidium and Giardia in natural surface waters in Finland.” J Microbiol Methods 50(3): 299-303.
Rimhanen-Finne, R., P. Ronkainen, et al. (2001). “Simultaneous detection of Cryptosporidium parvum and Giardia in sewage sludge by IC-PCR.” J Appl Microbiol 91(6): 1030-5.
Rimhanen-Finne, R., P. Ronkainen, et al. (2001). “Simultaneous detection of Cryptosporidium parvum and Giardia in sewage sludge by IC-PCR.” J Appl Microbiol 91(6): 1030-5.
Ripabelli, G., A. Leone, et al. (2004). “Detection of Cryptosporidium parvum oocysts in experimentally contaminated lettuce using filtration, immunomagnetic separation, light microscopy, and PCR.” Foodborne Pathog Dis 1(4): 216-22.
Rochelle, P. A., R. De Leon, et al. (1997). “Comparison of primers and optimization of PCR conditions for detection of Cryptosporidium parvum and Giardia lamblia in water.” Appl Environ Microbiol 63(1): 106-14.
Rochelle, P. A., R. De Leon, et al. (1997). “Comparison of primers and optimization of PCR conditions for detection of Cryptosporidium parvum and Giardia lamblia in water.” Appl Environ Microbiol 63(1): 106-14.
Rochelle, P. A., D. M. Ferguson, et al. (1996). “Development of a rapid detection procedure for Cryptosporidium, using in vitro cell culture combined with PCR.” J Eukaryot Microbiol 43(5): 72S.
Rochelle, P. A., D. M. Ferguson, et al. (1997). “An assay combining cell culture with reverse transcriptase PCR to detect and determine the infectivity of waterborne Cryptosporidium parvum.” Appl Environ Microbiol 63(5): 2029-37.
Schroeder, A. A., A. M. Brown, et al. (1998). “Identification and cloning of a developmentally regulated Cryptosporidium parvum gene by differential mRNA display PCR.” Gene 216(2): 327-34.
Shen, Y. J., J. P. Cao, et al. (2005). “[Preparation of DNA from Cryptosporidium parvum oocysts for PCR detection].” Zhongguo Ji Sheng Chong Xue Yu Ji Sheng Chong Bing Za Zhi 23(4): 228-30.
Shianna, K. V., R. Rytter, et al. (1998). “Randomly amplified polymorphic DNA PCR analysis of bovine Cryptosporidium parvum strains isolated from the watershed of the Red River of the North.” Appl Environ Microbiol 64(6): 2262-5.
Sousa, M. C., J. B. Morais, et al. (2006). “Genotyping of Giardia lamblia human isolates from Portugal by PCR-RFLP and sequencing.” J Eukaryot Microbiol 53 Suppl 1: S174-6.
Spano, F., L. Putignani, et al. (1998). “Cryptosporidium parvum: PCR-RFLP analysis of the TRAP-C1 (thrombospondin-related adhesive protein of Cryptosporidium-1) gene discriminates between two alleles differentially associated with parasite isolates of animal and human origin.” Exp Parasitol 90(2): 195-8.
Spano, F., L. Putignani, et al. (1997). “PCR-RFLP analysis of the Cryptosporidium oocyst wall protein (COWP) gene discriminates between C. wrairi and C. parvum, and between C. parvum isolates of human and animal origin.” FEMS Microbiol Lett 150(2): 209-17.
Stinear, T., A. Matusan, et al. (1996). “Detection of a single viable Cryptosporidium parvum oocyst in environmental water concentrates by reverse transcription-PCR.” Appl Environ Microbiol 62(9): 3385-90.
Stinear, T., A. Matusan, et al. (1997). “Detection of a Single Viable Cryptosporidium parvum Oocyst in Environmental Water Concentrates by Reverse Transcription-PCR.” Appl Environ Microbiol 63(2): 815.
Stroup, S. E., S. Roy, et al. (2006). “Real-time PCR detection and speciation of Cryptosporidium infection using Scorpion probes.” J Med Microbiol 55(Pt 9): 1217-22.
Sturbaum, G. D., P. T. Klonicki, et al. (2002). “Immunomagnetic separation (IMS)-fluorescent antibody detection and IMS-PCR detection of seeded Cryptosporidium parvum oocysts in natural waters and their limitations.” Appl Environ Microbiol 68(6): 2991-6.
Sturbaum, G. D., C. Reed, et al. (2001). “Species-specific, nested PCR-restriction fragment length polymorphism detection of single Cryptosporidium parvum oocysts.” Appl Environ Microbiol 67(6): 2665-8.
Tanriverdi, S., A. Tanyeli, et al. (2002). “Detection and genotyping of oocysts of Cryptosporidium parvum by real-time PCR and melting curve analysis.” J Clin Microbiol 40(9): 3237-44.
Tian, Z. C., X. C. Zhang, et al. (2002). “[Cloning of a species-specific gene fragment from Cryptosporidium parvum and the development of diagnostic PCR primers].” Zhongguo Ji Sheng Chong Xue Yu Ji Sheng Chong Bing Za Zhi 20(2): 72-5.
Verweij, J. J., R. A. Blange, et al. (2004). “Simultaneous detection of Entamoeba histolytica, Giardia lamblia, and Cryptosporidium parvum in fecal samples by using multiplex real-time PCR.” J Clin Microbiol 42(3): 1220-3.
Verweij, J. J., R. A. Blange, et al. (2004). “Simultaneous detection of Entamoeba histolytica, Giardia lamblia, and Cryptosporidium parvum in fecal samples by using multiplex real-time PCR.” J Clin Microbiol 42(3): 1220-3.
Verweij, J. J., J. Schinkel, et al. (2003). “Real-time PCR for the detection of Giardia lamblia.” Mol Cell Probes 17(5): 223-5.
Wagner-Wiening, C. and P. Kimmig (1995). “Detection of viable Cryptosporidium parvum oocysts by PCR.” Appl Environ Microbiol 61(12): 4514-6.
Walker, M. J., C. Montemagno, et al. (1998). “Method detection limits of PCR and immunofluorescence assay for Cryptosporidium parvum in soil.” Appl Environ Microbiol 64(6): 2281-3.
Ward, L. A. and Y. Wang (2001). “Rapid methods to isolate Cryptosporidium DNA from frozen feces for PCR.” Diagn Microbiol Infect Dis 41(1-2): 37-42.
Widmer, G. (1998). “Genetic heterogeneity and PCR detection of Cryptosporidium parvum.” Adv Parasitol 40: 223-39.
Wu, Z., I. Nagano, et al. (2003). “Intraspecies polymorphism of Cryptosporidium parvum revealed by PCR-restriction fragment length polymorphism (RFLP) and RFLP-single-strand conformational polymorphism analyses.” Appl Environ Microbiol 69(8): 4720-6.
Wu, Z., I. Nagano, et al. (2000). “Specific PCR primers for Cryptosporidium parvum with extra high sensitivity.” Mol Cell Probes 14(1): 33-9.
Xiao, L., A. A. Lal, et al. (2004). “Detection and differentiation of Cryptosporidium oocysts in water by PCR-RFLP.” Methods Mol Biol 268: 163-76.
Yong, T., K. Han, et al. (2002). “PCR-RFLP analysis of Giardia intestinalis using a Giardia-specific gene, GLORF-C4.” Parasite 9(1): 65-70.
Zhou, L., C. Yang, et al. (2003). “PCR-mediated recombination between Cryptosporidium spp. of lizards and snakes.” J Eukaryot Microbiol 50 Suppl: 563-5.
Zhou, R., G. Li, et al. (2007). “PCR amplification and sequence analyses of ITS-1 rDNA from Cryptosporidium andersoni in dairy cattle.” Parasitol Res 100(5): 1135-8.
Ziegler, P. E., F. Santucci, et al. (2007). “Evaluation of PCR Diagnosis of Cryptosporidium spp In Dairy Cattle and Wildlife.” Vet Ther 8(2): 148-59.
A-Z of Quantitative PCR, Stehen A. Bustin, editor, International University Line (publishers) 2004, ISBN—13:9780963681782.
U.S. Pat. No. 7,312,039—Barany; Francis, et al. (2007).
U.S. Pat. No. 7,081,527—Cunningham et al. (2006).
U.S. Pat. No. 6,867,021—Maes et al. (2005).
U.S. Pat. No. 6,436,638—DeLeon et al. (2002).
U.S. Pat. No. 6,395,517—Abbaszadegan et al. (2002).
U.S. Pat. No. 6,153,411—Abbaszadegan et al. (2002).
U.S. Pat. No. 6,146,855—Williams et al. (2000).
U.S. Pat. No. 5,770,368—DeLeon et al. (1998).
U.S. Pat. No. 5,558,989—Shah et al. (1996).
U.S. Pat. No. 5,756,701—Wu et al. (1998).
U.S. Pat. No. 7,294,466—McMillan et al. (2007).
U.S. Pat. No. 6,908,739—Buechler et al. (2005).

What is claimed is:

1. A nucleic acid-based method for simultaneously screening or detecting the presence of two or more microscopic pathogens in a sample, said method comprising:
isolating nucleic acids consisting of DNA of Cryptosporidium and Giardia from the sample to provide an isolate;
combining said isolate with a PCR reaction mixture and a combination of primer nucleic acid sequences and probe nucleic acid sequences that bind to a target Cryptosporidium nucleic acid sequence, primer nucleic acid sequences and probe nucleic acid sequences that bind to a target Giardia nucleic acid sequence, and an internal control construct;
amplifying target nucleic acid sequence in said isolate that bind said primer and probe nucleic acid sequences and;
detecting nucleic acid in the reaction mixture bound to amplified target nucleic acid sequence.
wherein the presence of amplified target nucleic acid sequences bound to probe nucleic acid sequences identifies the presence of Giardia and Cryptosporidium in the isolate from the sample.

2. The method of claim 1 wherein the primer nucleic acid sequences are:
Giardia Forward (primer 1):
(SEQ ID NO: 3)
5′-GGA CGG CTC AGG ACA AC-3′;
Giardia Reverse (primer 2):
(SEQ ID NO: 5)
5′-GGA GTC GAA CCC TGA TTC T-3′;
Cryptosporidium Forward (primer 1):
(SEQ ID NO: 2)
5′-GCC TAC CGT GGC AAT GA-3′;
Cyptosporidium Reverse (primer 2):
(SEQ ID NO: 4)
5′-AAA GTC CTG TAT TGT TAT TTC TTG TC-3′

3. The method of claim 1 wherein the probe nucleic acid sequences for Giardia are:
Giardia Probe 1:
(SEQ ID. NO. 8)
5′-CGT GAC GCA GCG ACG G-Fluorescein-3′;
Giardia Probe 2:
(SEQ ID NO: 9)
5′-LCRed705-CGC CCG GGC TTC CGG-Phosphate-3′.

4. The method of claim 1 wherein the probe nucleic acid sequences for Cryptosporidium are:
Cryptosporidium Probe 1:
(SEQ ID NO: 10)
5′-CGG CTA CCA CAT CTA AGG AAG GC-Fluorescein-3′;
Cryptosporidium Probe 2:
(SEQ ID NO: 11)
5′-LCRed640-CAG GCG CGC AAA TTA CCC AAT CCT A-
Phosphate-3′

5. The method of claim 1 wherein the PCR reaction mixture further comprises primers and probes that bind to an internal control template nucleic acid sequence.

6. The method of claim 1 wherein the target nucleic acid sequence in the isolate of the test sample is detected using a thermocycler via detection of fluorescent light excitation emitted by target nucleic acid sequence bound to fluorescently labeled Giardia, Cryptosporidium or both Giardia and Cryptosporidium nucleic acid sequence.

7. The method of claim 6 wherein Giardia is identified by a fluorophore emitting at a detectable wavelength of about 705 (high Red), and wherein Cryptosporidium is identified by a fluorophore emitting at a detectable wavelength of about 635 (Red).

8. The method of claim 1 wherein the sample is a water sample or a stool sample.

9. The method of claim 8 wherein the stool sample is a human stool sample.

10. The method of claim 5 wherein the internal control construct comprises a sequence:
(SEQ ID NO: 7)
5′- GCC TAC CGT GGC AAT GAA GTT AGT AGT GCG ATC
CTT TCT GAC TTT TGT CGT GCT GTG ACG GTG CTT GCC
ATG CGA ACA GCT GCA CAG GTA CTC GAG GGA AGG CAC
GTA AAT TTA GTC CCC CAA TAA ATA ACA GGC CGC TGT
TGA GCA CAA GCA GCT AGC GCC GTT TTA GCC ACA TGT
ACC CAG TAT ATA TGT CAC GAG AGG ATA GGC GAA TTG
GAA TGG TCA GGC CGA CAA GAA ATA ACA ATA CAG GAC
TTT -3′

11. A kit for screening a sample for two or more biological contaminants comprising:
two or more primer nucleic acid sequences, at least one of said primer nucleic acid sequences being specific for Giardia and at least one of said primer nucleic acid sequences being specific for Cryptosporidium;
two or more probe nucleic acid sequences, at least one of said probe nucleic acid sequences being specific for Giardia and at least one of said probe nucleic acid sequences being specific for Cryptosporidium; and
an internal control construct.

12. The kit of claim 11 wherein the kit comprises an instructional manual.

13. A method of screening to simultaneously detect Cryptosporidium parvum and Giardia in a human fecal sample, the method comprising:
(a) isolating nucleic acid from a human fecal sample to provide a sample nucleic acid isolate;
(b) mixing the sample nucleic acid isolate in a PCR reaction mixture comprising:
a first fluorophore labeled oligonucleotide primer pair consisting of an upstream primer having a nucleic acid sequence of SEQ ID NO: 2 and a downstream primer having a nucleic acid sequence of SEQ ID NO: 4, said primers being capable of annealing to a first target nucleic acid sequence of Cryptosporidium parvum,
a second fluorophore labeled oligonucleotide primer pair consisting of an upstream primer having a nucleic acid sequence of SEQ ID NO: 3 and a downstream primer having a nucleic acid sequence of SEQ ID NO: 5, said primers being capable of annealing to a second target nucleic acid sequence of Giardia;
a third oligonucleotide probe pair specific for Giardia;
a fourth oligonucleotides probe pair specific for Cryptosporidium;
an internal control (IC) construct nucleic acid sequence comprising a sequence of SEQ ID NO: 1;
and
four deoxynucleotide triphosphates selected from the group consisting of adenosine deoxynucleotide triphosphate, guanosine deoxynucleotide triphosphate, thymidine deoxynucleotide triphosphate, cytosine deoxynucleotide triphosphate, and nucleotide analogs thereof;
(c) providing a thermostable DNA polymerase;
(d) amplifying by a PCR reaction the first target nucleic acid from the DNA of the Cryptosporidium parvum and the second target nucleic acid from the Giardia DNA, in the reaction mixture under suitable PCR reaction mixture temperature conditions by a repetitive series of PCR thermal cycling steps comprising:
(1) denaturing the DNA into denatured strands;
(2) annealing the oligonucleotide primers provided in step (b) to the denatured strands of the DNA;
(3) extending the hybridized primers with the four deoxynucleotide triphosphates and the nucleic acid polymerase to provide amplified PCR products; and
(4) following amplification, screening for the first and second target nucleic acids in the amplified PCR products so as to simultaneously detect the Cryptosporidium parvum and Giardia, respectively, in the human fecal sample.

14. The method of claim 13 wherein the PCR reaction is for 40-50 cycles wherein each cycle consists of denaturing at about 95° C. for about 10-30 seconds, annealing at 50°-60° C. for about 10-30 seconds, and extending at about 72° C. for about 20-30 seconds.

15. The method of claim 13 wherein the primer pair specific for Cryptosporidium are selected from the group of primer pairs consisting of:
Cryptosporidium Forward (primer1):
(SEQ ID NO: 2)
5′- GCC TAC CGT GGC AAT GA-3′;
Cryptosporidium Reverse (primer2):
(SEQ ID NO: 4)
5′- AAA GTC CTG TAT TGT TAT TTC TTG TC-3′

16. The method of claim 13 wherein the sample comprises the Cryptosporidium and the Giardia which are isolated from a human fecal sample by suspension in lysis buffer and subsequent DNA extraction.

17. The method of claim 13 that includes one or more probes for detecting the amplified PCR product wherein each probe is complementary to a sequence within the target sequence of Cryptosporidium parvum and Giardia.

18. The method of claim 17 wherein the probes are labeled at its 5′ end with a fluorosceine and labeled at its 3′ end with a phosphate.

19. The method of claim 18 wherein the probes are blocked against chain extension at its 3′ end.

20. An internal control construct (ICC) comprising a structure: 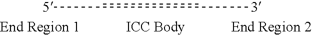
wherein said construct comprises an ICC body, an end region 1 and an end region 2.

21. The internal control construct of claim 20 wherein the end region 1 and the end region 2 may comprise the same or different base pair sequences.

22. The internal control construct of claim 20 wherein the end region 1 and end region 2 comprise a sequence that corresponds to the base pair sequence of a primer sequence of a target microorganism, and wherein each of the end region 1 and the end region 2 posses a length of 15 bp to 30 bp.


SEQUENCE LISTING (12 sequences)

SEQ ID NO: 1 - DNA, 211 bp, Artificial Sequence
  Description of Artificial Sequence Synthetic construct
gaagttagtagtgcgatcctttctgacttttgtcgtgctgtgacggtgcttgccatgcgaacagctgcacaggtactcgagggaaggcacgtaaatttagtcccccaataaataacaggccgctgttgagcacaagcagctagcgccgttttagccacatgtacccagtatatatgtcacgagaggataggcgaattggaatggtcaggcc

SEQ ID NO: 2 - DNA, 17 bp, Artificial Sequence
  Description of Artificial Sequence Synthetic primer
gcctaccgtggcaatga

SEQ ID NO: 3 - DNA, 17 bp, Artificial Sequence
  Description of Artificial Sequence Synthetic primer
ggacggctcaggacaac

SEQ ID NO: 4 - DNA, 26 bp, Artificial Sequence
  Description of Artificial Sequence Synthetic primer
aaagtcctgtattgttatttcttgtc

SEQ ID NO: 5 - DNA, 19 bp, Artificial Sequence
  Description of Artificial Sequence Synthetic primer
ggagtcgaaccctgattct

SEQ ID NO: 6 - DNA, 30 bp, Artificial Sequence
  Description of Artificial Sequence Synthetic probe
gtcgtgctgtgacggtgcttgccatgcgaa

SEQ ID NO: 7 - DNA, 252 bp, Artificial Sequence
  Description of Artificial Sequence Synthetic construct
gcctaccgtggcaatgaagttagtagtgcgatcctttctgacttttgtcgtgctgtgacggtgcttgccatgcgaacagctgcacaggtactcgagggaaggcacgtaaatttagtcccccaataaataacaggccgctgttgagcacaagcagctagcgccgttttagccacatgtacccagtatatatgtcacgagaggataggcgaattggaatggtcaggccgacaagaaataacaatacaggacttt

SEQ ID NO: 8 - DNA, 16 bp, Artificial Sequence
  Description of Artificial Sequence Synthetic probe
cgtgacgcagcgacgg

SEQ ID NO: 9 - DNA, 15 bp, Artificial Sequence
  Description of Artificial Sequence Synthetic probe
cgcccgggcttccgg

SEQ ID NO: 10 - DNA, 23 bp, Artificial Sequence
  Description of Artificial Sequence Synthetic probe
cggctaccacatctaaggaaggc

SEQ ID NO: 11 - DNA, 25 bp, Artificial Sequence
  Description of Artificial Sequence Synthetic probe
caggcgcgcaaattacccaatccta

SEQ ID NO: 12 - DNA, 30 bp, Artificial Sequence
  Description of Artificial Sequence Synthetic probe
agctgcacaggtactcgagggaaggcacgt